(19) United States
(12) Patent Application Publication
(10) Pub. No.: US 2009/0227960 A1
(43) Pub. Date: Sep. 10, 2009

(54) Flow Control Assembly Including a Valve and Flow Controller

(76) Inventor: Zoran Milijasevic, New South Wales (AU)

Correspondence Address: NELSON MULLINS RILEY & SCARBOROUGH, LLP, 1320 MAIN STREET, 17TH FLOOR, COLUMBIA, SC, 29201, US

(21) Appl. No.: 12/086,196

(22) PCT Filed: Nov. 27, 2006
(86) PCT No.: PCT/AU2006/001792

§ 371 Date: Apr. 3, 2009

(30) Foreign Application Priority Data
Dec. 12, 2005 (AU) 2005906976

(51) Int. Cl. A61M 5/168

(52) U.S. Cl. 604/246

(57) ABSTRACT

A flow control assembly (10) includes a housing (12) having an upstream end (14) which communicates with a source of fluid to be dispensed, in use, and a downstream end (16) which communicates with a supply conduit (26)connectable to the downstream end (16). A passage (18) is defined through the housing (12) between the upstream end (14) and the downstream end (16) of the housing (12). A valve arrangement (20) is arranged in the passage (18) for controlling the flow of fluid through the passage (18). A flow controller (21) is associated with the valve arrangement (20). The flow controller (21) facilitates flow of fluid through the passage (18) regardless of a state of the valve arrangement (20).

### CROSS-REFERENCE TO RELATED APPLICATIONS

The present application claims priority from Australian Provisional Patent Application No 2005906976 filed on 12 Dec. 2005, the contents of which are incorporated herein by reference.

### FIELD OF THE INVENTION

This invention relates, generally, to a flow control assembly and, more particularly but not necessarily exclusively, to a flow control assembly for an intravenous (IV) administration set.

### BACKGROUND TO THE INVENTION

IV administration sets are commonly used for infusing fluids into a patient's body. The flow rate of fluid through the administration set from a reservoir of the fluid is governed by a pressure head. The pressure head is generated by having the reservoir arranged at an elevated location relative to the entry point of fluid into the patient's body and/or by use of a pressure generating device such as a pump.

It is necessary to maintain the infusion rate of the fluid at a predetermined rate to ensure that the patient receives the correct dosage of the fluid being dispensed.

There are three main factors controlling the infusion rate of the fluid. These are, firstly, the pressure head created, secondly, the friction or resistance to flow in the system and, thirdly, the viscosity or density of the fluid being infused.

The pressure head obtained by an elevated reservoir may change due to the back pressure. The back pressure created can cause changes in the flow rate of the fluid and the pressure may change inadvertently, for example, by a patient turning over in bed or by standing up. Viscosity of the fluid may change due to ambient factors such as changes in the ambient temperature, etc.

It has been proposed to use an orifice plate to obtain a constant flow rate of the fluid to be infused independent of the viscosity of the fluid being infused. However, a needle assembly of an IV administration set is also sometimes used for injecting fluids into a patient. An orifice plate downstream of the injection point may impede injection of the fluid due to the small dimensions of the orifice. Also, there is the danger of back flow of bodily fluids from the patient's body when the administration set is removed. This bodily fluid may come into contact with the person removing the set and could have adverse consequences due to blood borne diseases and the like.

### SUMMARY OF THE INVENTION

According to the invention, there is provided a flow control assembly, the flow control assembly including

a housing having an upstream end which communicates with a source of fluid to be dispensed, in use, and a downstream end which communicates with a supply conduit connectable to the downstream end with a passage being defined through the housing between the upstream end and the downstream end of the housing;

a valve arrangement arranged in the passage for controlling the flow of fluid through the passage; and

a flow controller associated with the valve arrangement, the flow controller facilitating flow of fluid through the passage regardless of a state of the valve arrangement.

In this specification, the term “state of the valve arrangement” means, unless the context clearly indicates otherwise, whether or not the valve arrangement is open or closed.

The housing may be connectable to, or form part, of an outlet of a reservoir of the fluid. The reservoir may function as the source of fluid. Instead, the housing may. be mounted in-line in the supply conduit or form part of the supply conduit. Further, the upstream end of the housing may be shaped to receive an end of a syringe. Thus, the upstream end of the housing may be in the form of a female component of a Luer lock.

The valve arrangement may comprise at least two valves arranged in series with a first valve of the series being an upstream valve and a second valve of the series being a downstream valve. Each valve may have an operating member which opens at a predetermined fluid pressure. The arrangement may be such that the downstream valve opens at a substantially lower pressure than the upstream valve.

The operating member of at least the first valve may comprise a pair of opposed leaves which are arranged in a normally closed configuration to form a duck bill valve.

At least the upstream valve may have the flow controller associated with it. The flow controller may comprise an orifice plate. The orifice plate may be mounted in one of the leaves of the operating member of the upstream valve. It will be appreciated-that the orifice plate serves to control the flow rate of the fluid through the passage in a manner substantially independently of the viscosity of the fluid.

The operating member of the second valve may be configured to open at a pressure caused only by the passage of fluid through the orifice plate. Further, the second valve may be tailored to open at a pressure dependent on the desired rate at which the fluid is to be infused.

The first valve of the valve arrangement may be configured to open when a fluid is dispensed from a dispenser other than the reservoir, the fluid being dispensed at a rate substantially higher than the flow rate governed by the orifice plate.

The second valve of the valve arrangement may inhibit back flow of fluid in the conduit and therefore reduce the risk of users of a system incorporating the flow control assembly coming into contact with bodily fluids of a patient.

The invention extends also to an intravenous administration set which includes a flow control assembly as described above.

### BRIEF DESCRIPTION OF THE DRAWINGS

FIG. 1 shows a schematic, sectional side view of a flow control assembly in accordance with an embodiment of the invention.

### DETAILED DESCRIPTION OF EXEMPLARY EMBODIMENT

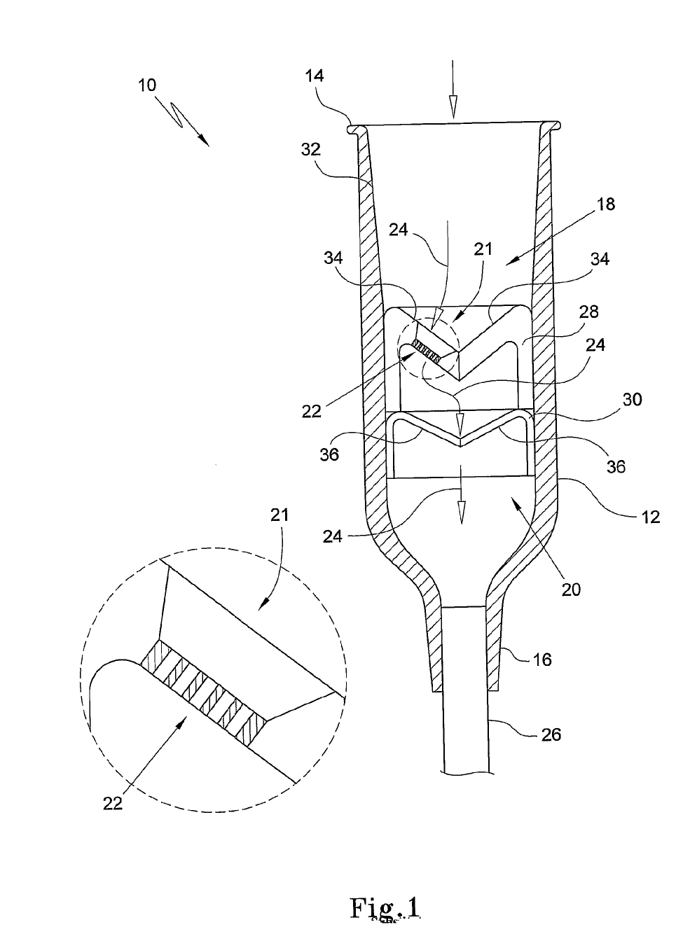

In FIG. 1, reference numeral 10 generally designates a flow control assembly in accordance with an embodiment of the invention. While the assembly 10 may have uses in other applications, it will be described below with reference to its application in an intravenous (IV) administration set.

The flow control assembly comprises an elongate housing 12 having an upstream end 14 and a downstream end 16. A passage 18 is defined. through the housing 12.

A valve arrangement 20 is arranged in the passage 18 of the housing 12 intermediate the upstream end 14 and the downstream end 16 of the housing 12.

A flow controller 21, comprising an orifice plate 22, is associated with the valve arrangement 20. The flow controller 21 facilitates flow of a fluid, or liquid, such as a saline solution, through the passage 18 regardless of a state of the valve arrangement 20.

The upstream end 14 is connectable to a source of liquid (not shown). Generally, the assembly 10 is used with an IV administration set and the source of liquid is a reservoir of the liquid which is to be infused at a predetermined flow rate into a patient's body. This is achieved by the liquid flowing through the orifice plate 22 as indicated by arrows 24.

The downstream end 16 of the housing 12 mounts a supply conduit 26. The supply conduit 26 leads to a needle assembly (not shown), a needle of which is inserted into a vein of the patient's body for the infusion of the liquid.

The valve arrangement 20 comprises a first valve 28 and a second valve 30 arranged downstream of the first valve 28.

The first valve 28 is configured to open at a predetermined pressure of liquid upstream of the valve 28. The valve 28 is designed to open when liquid is to be injected into the patient from a source other than the reservoir of the IV administration set, for example, when liquid is to be injected from a syringe.

For this purpose, the upstream end 14 of the housing 12 defines a female Luer lock formation 32 into which a male Luer lock of the syringe is receivable for injecting the liquid into the patient.

The valve 28 has a pair of operating members 34 arranged in a duck-bill formation. The orifice plate 22 is arranged in one of the operating members 34 so that liquid at the required infusion flow rate can pass through the housing 12 regardless of the state of the valve 28, i.e. regardless of whether the valve 28 is in an open configuration or a closed configuration.

The second valve 30 has a pair operating members 36 also arranged in a duck-bill configuration, which are configured to open at an extremely low pressure and a pressure significantly lower than the pressure at which the operating members 34 of the valve 28 open. More specifically, the operating members 36 of the valve 30 are configured to open at fluid pressures corresponding to the infusion flow rate of the liquid through the orifice plate 22. Further, the operating members 36 of the valve 30 can be tailored to open at different infusion flow rates.

The purpose of the valve 30 is to inhibit backflow of fluids, such as blood, when the administration set is removed from the assembly 10.

In use, the upstream end 14 of the housing 12 is connected to an outlet of the reservoir of liquid to be infused via an administration set. After suitable priming, liquid passes through the orifice plate 22 of the flow controller 21 and causes the operating member 36 of the valve 30 to open so that liquid passes into the conduit 26 and, via the needle assembly, into the patient's body at the required flow rate.

When it is required to inject a substance into the patient, the housing 12 is removed from the outlet of the reservoir and the male Luer lock of the syringe is inserted into the upstream end 14 of the housing 12. Expulsion of the liquid from the syringe causes the valve 28 to open and, as a result, also the valve 30. The liquid can thus be injected at a much higher rate than the flow rate through the flow controller 21.

After completion of the injection of the liquid, the housing 12 is reattached to the reservoir to allow continued infusion of the liquid from the reservoir.

When the needle assembly is to be removed from the patient's body, the supply of liquid from the reservoir is, firstly, stopped. This may create a low pressure region upstream of the valve 30. The valve 30 remains closed retaining pressure in the supply conduit 26. Thus, when the male Luer assembly of the IV administration set is removed from the assembly 10, the back flow of bodily fluids from the patient is inhibited.

It is a first advantage of the invention that a flow control assembly 10 is provided which facilitates the use of the same device, both for infusion and for injection of liquids. Secondly, the flow control assembly 10 inhibits the back flow of bodily fluids from a patient's body thereby inhibiting the chances of contamination of a user by such bodily fluids.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A flow control assembly, the flow control assembly including
a housing having an upstream end which communicates with a source of fluid to be dispensed, in use, and a downstream end which communicates with a supply conduit connectable to the downstream end with a passage being defined through the housing between the upstream end and the downstream end of the housing;
a valve arrangement arranged in the passage for controlling the flow of fluid through the passage; and
a flow controller associated with the valve arrangement, the flow controller facilitating flow of fluid through the passage regardless of a state of the valve arrangement.

2. The assembly of claim 1 in which the valve arrangement comprises at least two valves arranged in series with a first valve of the series being an upstream valve and a second valve of the series being a downstream valve.

3. The assembly of claim 2 in which each valve has an operating member which opens at a predetermined fluid pressure with the downstream valve opening at a substantially lower pressure than the upstream valve.

4. The assembly of claim 2 in which the operating member of at least the first valve comprises a pair of opposed leaves which are arranged in a normally closed configuration to form a duck bill valve.

5. The assembly of any one of claim 2 in which at least the upstream valve has the flow controller associated with it.

6. The assembly of claim 5 in which the flow controller comprises an orifice plate mounted in the operating member of the upstream valve.

7. The assembly of claim 6 in which the operating member of the second valve is configured to open at a pressure caused only by the passage of fluid through the orifice plate.

8. The assembly of claim 7 in which the second valve is tailored to open at a pressure dependent on the desired rate at which the fluid is to be infused.

9. The assembly of any one of claim 6 in which the first valve of the valve arrangement is configured to open when a fluid is dispensed through the housing from a dispenser other than a reservoir to which the housing is connected in use, the fluid being dispensed at a rate substantially higher than the flow rate governed by the orifice plate.

10. An intravenous administration set which includes a flow control assembly as claimed in claim 1.
